(19) United States
(12) Patent Application Publication
(10) Pub. No.: US 2022/0259553 A1
(43) Pub. Date: Aug. 18, 2022

(54) SYSTEMS AND METHODS FOR COLLECTING BIOAEROSOLS

(71) Applicant: SPORNADO INC., Toronto (CA)

(72) Inventors: Michael SALEH, Toronto (CA); Kristine WHITE, Toronto (CA); James SCOTT, Toronto (CA)

(21) Appl. No.: 17/610,782

(22) PCT Filed: Dec. 23, 2019
(86) PCT No.: PCT/CA2019/051901

§ 371 Date: Nov. 12, 2021

Related U.S. Application Data
(60) Provisional application No. 62/848,441, filed on May 15, 2019.

(51) Int. Cl. C12N 3/00; G01N 1/22; C12Q 1/6895; C12Q 3/00

(52) U.S. Cl. CPC C12N 3/00; C12Q 3/00; C12Q 1/6895; G01N 1/2273

(57) ABSTRACT

Devices for collecting bioaerosols are provided, including a cassette comprising a mesh made of electrostatically charged fibers held taut between a pair of mating members for supporting the collection medium in a extended position to expose a capture surface for capturing bioaerosols. The cassette is replaceably inserted in a wind vane apparatus which directs airflow to the capture surface of the cassette for capturing bioaerosols.

### FIELD

The present disclosure generally relates to the field of agricultural surveillance, including systems and methods for collecting and analyzing bioaerosols.

### BACKGROUND

Plant diseases are one of the main causes of crop loss, which in turn leads to economic loss, food shortage, and loss of viable crop for future propagation. Pathogens are one of the three factors to crop disease, the other two being host susceptibility and environment conditions.

To combat plant diseases caused by pathogens, pesticides are applied to crops. However, the application of pesticides is typically based on grower experience combined with review of modelling predictions for a region based on environmental factors such as the weather, if available.

Thus, there remains a need for improved systems, devices, and methods for gathering pathogen information to generate pesticide use decisions.

### SUMMARY

In one aspect, there is provided a passive particulate capture device and system for passively collecting bioaerosols, such as pathogens or spores, without using a motorized pump.

In one aspect an improved passive sampling device is provided that is easy to use for farmers and growers in addition to and researchers. The improved passive sampling device is cheaper to manufacture which in turn allows farmers and growers to place the devices in individual fields and to obtain localized data.

In another aspect, there is provided a replaceable cassette for capturing bioaerosols.

In another aspect, there is provided a pathogen collection system comprising a cassette and a wind vane apparatus.

In another aspect, there is provided a method of monitoring crops by capturing pathogen using the cassettes and collection systems described herein, and detecting the presence and/or absence of target bioaerosols, including pathogens.

Many further features and combinations thereof concerning embodiments described herein will appear to those skilled in the art following a reading of the instant disclosure.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

### DESCRIPTION OF THE FIGURES

Embodiments of devices, apparatus, and methods are described throughout reference to the drawings.

FIG. 1 is a perspective view of a cassette for capturing bioaerosols.

FIG. 2 is a side view of the cassette of FIG. 1.

FIG. 3 is a front view of the cassette of FIG. 2.

FIG. 4 is a perspective view of a wind vane apparatus. Arrow indicates direction of airflow.

FIG. 5 is a first perspective view of a wind vane apparatus loaded with a cassette.

FIG. 6 is a second perspective view of FIG. 5.

FIG. 7 is a flow diagram showing step involved in providing pesticide spray decisions.

### DETAILED DESCRIPTION

Passive collection of particulates from an air stream using a passive sampling device to capture bioaerosols including potential pathogens has numerous advantages over currently existing devices that actively drawing air onto a medium using a mechanical pump (referred to as volumetric sampling devices). Volumetric spore trap sampling is used in a wide variety of applications for epidemiological, health and safety settings, but only on a limited scale in agricultural, mostly research-based because commercially available technologies have been cost prohibitive and not easy to use. However, volumetric sampling devices are expensive, require regular maintenance as well as a power supply, such as a power generator, which is cumbersome and vulnerable to weather when the volumetric sampling device is placed in a crop field.

A passive sampling device requires less expensive components, and can be easily placed throughout a crop field since no power source is needed. At the same time, a passive sampling device draws in less air than one powered by a mechanical pump, and hence less particulate matter, such as pathogens, passes through. Therefore, improved pathogen capture devices and a highly sensitive method of sample analysis are required to optimize passive sampling.

Bioaerosols Capture

One existing system uses indoor air sampler and a cassette, containing a slide for microscopic identification. It provides a short term “snap shot” collecting a sample for only 5-15 min, during which the spores may not be present in the air. (See Canadian patent no. 2969282, the entire content of which is incorporated herein by reference.) This system currently uses microscopic ID, which is much less sensitive and relies on the training and skill of the analyst conducting the sampling. This system also lacks robustness and is not designed for other bioaerosols.

Other existing sampling devices include: Roto™ rod which has a sticky adhesive on an rod, which is messy, difficult to use, and lacks robustness; or Burkhard™ which is a very expensive equipment and difficult to use.

The present inventors has discovered that using a mesh material allows for optimally capture bioaerosols including crop pathogens, while also allowing for air flow and molecular analysis with minimal sample preparation. In some embodiments, a cassette comprising a mesh material for capture of bioaerosols is left in the field for several days (typically 3-7 days), providing long term sampling. Longer term sampling provides more integrated data compared to a snap shot approach. Spores in the air depend on a variety of factors (e.g. wind speed, weather conditions such as rain, time of day and time of year. A snap shot approach can be hit or miss while integrated long term sampling has the chance to sample during different conditions and increase probability of capturing target. Accordingly, cassettes are provided for long term sampling. In one embodiment, a pathogen capture device is provided having a medium made of fibers, preferably electrostatically charged fibers.

As used herein, “bioaerosols” refers to biological aerosols, which are tiny airborne particles that are biological in nature. Bioaerosols come from a living organism (such as dander from indoor pets or pollen from trees) or are living organisms themselves (such as bacteria and viruses). As used herein, “pathogen” refers to any matter that can cause disease. Pathogens that are present in the air include plant pathogens. In one embodiment, the pathogen capture device captures spores, fragments of spores, and/or hyphae.

In some embodiments of the device for capturing bioaerosols or pathogens, the bioaerosols or pathogens include, powdery mildew, downy mildew, botytris, fusarium, early blight, or apple scab.

In some embodiments of the device for capturing spores, the spores are from the plant pathogen Phytophthora. As used herein, the term “Phytophthora” includes all the species of the genus Phytophthora. The species of Phytophthora captured and/or can include any of Phytophthora taxon Agathis, Phytophthora alni, Phytophthora boehmeriae, Phytophthora botryose, ibrassicae, Phytophthora cactorum, Phytophthora cajani, Phytophthora cambivora, Phytophthora capsici, Phytophthora cinnamomi, Phytophthora citricola, Phytophthora citrophthora, Phytophthora clandestine, Phytophthora colocasiae, Phytophthora cryptogea, Phytophthora drechsleri, Phytophthora diwan ackerman, Phytophthora erythroseptica, Phytophthora fragariae, Phytophthora fragariae var. rubi, Phytophthora Gemini, Phytophthora glovera, Phytophthora gonapodyides, Phytophthora heveae, Phytophthora hibemalis, Phytophthora humicola, Phytophthora hydropathical, Phytophthora irrigate, Phytophthora idaei, Phytophthora ilicis, Phytophthora infestans, Phytophthora inflate, Phytophthora ipomoeae, Phytophthora iranica, Phytophthora katsurae, Phytophthora kemoviae, Phytophthora lateralis, Phytophthora medicaginis, Phytophthora megakarya, Phytophthora megasperma, Phytophthora melonis, Phytophthora mirabilis, Phytophthora multivesiculata, Phytophthora nemorosa, Phytophthora nicotianae, Phytophthora PaniaKara, Phytophthora palmivora, Phytophthora phaseoli, Phytophthora pini, Phytophthora porri, Phytophthora plurivora, Phytophthora primulae, Phytophthora pseudosyringae, Phytophthora pseudotsugae, Phytophthora quercina, Phytophthora ramorum, Phytophthora sinensis, Phytophthora sojae, Phytophthora syringae, Phytophthora tentaculata, Phytophthora trifolii or Phytophthora vignae.

In one embodiment the device captures spores from the plant pathogen Sclerotinia. As used herein, the term “Sclerotinia” includes all the species of the genus Sclerotinia. The species of Sclerotinia captured and/or can include any of Sclerotinia borealis, Sclerotinia bulborum, Sclerotinia homoeocarpa, Sclerotinia minor, Sclerotinia ricin, Sclerotinia sclerotiorum, Sclerotinia spermophila, Sclerotinia sulcata, Sclerotinia trifoliorum, or Sclerotinia veratri.

In some embodiments, the device captures pathogens derived from one or more of those listed in Table 1.

TABLE 1
Major fungal pathogens

Aecidium clematidis
Albugo candida
Alternaria alternate
Alternaria brassicae
Alternaria lini
Alternaria linicola
Alternaria raphani
Alternaria sp.
Ascochyta fabae
Ascochyta lentis
Ascochyta pisi
Ascochyta rabiei
Aureobasidium zeae
Bipolaris sorokiniana
Blumeria graminis
Botrytis cinerea
Ceratobasidium cereale
Cercospora sojina
Cercospora zeae-maydis
Cercosporidium/Scolicotrichum graminis
Cladosporium herbarum
Claviceps purpurea
Cochliobolus sativus
Collectotrichum trifolii
Colletotrichum graminicola
Colletotrichum lini
Colletotrichum truncatum
Coprinus psychromorbidus
Coprinus sp.
Diaporthe phaseolorum
Dilophospora alopecuri
Drechslera graminea
Epicoccum sp.
Erysiphe graminis
Erysiphe pisi
Fusarium avenaceum
Fusarium culmorum
Fusarium graminearum
Fusarium nivale
Fusarium oxysporum
Fusarium oxysporum f. sp. lini.
Fusarium pseudograminearum
Fusarium roseum
Fusarium sp.
Fusarium spp.
Gaeumannomyces graminis
Gibberella zeae
Helminthosporium sativum/Cochliobolus sativus
Hymenula cerealis/Cephalosporium gramineum
Leptosphaeria biglobosa
Leptosphaeria maculans
Leptosphaerulina trifolii
Leptotrochila medicaginis
Macrophomina phaseolina
Melampsora lini
Microdochium/Fusarium nivale
Microsphaera diffusa
Monographella nivalis
Mycoleptodiscus sp.
Mycosphaerella graminicola
Mycosphaerella pinodes
Mycosphaerella tassiana
Myriosclerotinia/Sclerotinia borealis
Oidium lini
Peronospora trifoliorum
Peronospora viciae
Peronspora parasitica
Phaeosphaeria/Leptosphaeria herpotrichoides
Phakopsora pachyrhizi
Phoma medicaginis.
Phytophthora megasperma f. sp. medicaginis
Polyspora lini
Pseudocercosporella capsellae
Pseudocercosporella herpotrichoides
Pseudoseptoria/Selenophoma donacis
Psuedopeziza medicaginis
Puccinia coronata f. sp. avenae
Puccinia graminis
Puccinia graminis f. sp. avenae
Puccinia graminis f. sp. secalis
Puccinia graminis f. sp. tritici
Puccinia helianthi
Puccinia hordei
Puccinia recondita
Puccinia sorghi
Puccinia striiformis
Puccinia striiformis f. sp. tritici
Puccinia triticina
Pyrenophora graminea
Pyrenophora teres
Pyrenophora tritici-repentis
Pythium aphanidermatum
Pythium arrhenomanes
Pythium debaryanum
Pythium graminicola
Pythium irregulare
Pythium sp.
Pythium ultimum
Pythoum sp.
Rhizoctonia cerealis
Rhizoctonia solani
Rhynchosporium secalis
Sclerotinia borealis
Sclerotinia sclerotiorum
Septoria glycines
Septoria linicola
Septoria passerinii
Septoria secalis
Septoria tritici
Setosphaeria turcica
Sphacelia segetum
Sporobolomyces sp.
Stagonospora avenae
Stagonospora nodorum
Stagonospora/Septoria/Phaeosphaeria/Leptosphaeria nodorum
Stemphylium botryosum
Stemphylium sp.
Tapesia acuformis
Tilletia controversa
Tilletia indica
Tilletia laevis/foetida
Tilletia tritici/caries
Tilletia/Neovossia indica
Uredo glumarum
Ustilago hordei
Ustilago nigra
Ustilago nuda
Ustilago tritici
Verticillium albo-atrum
Verticillium longisporum

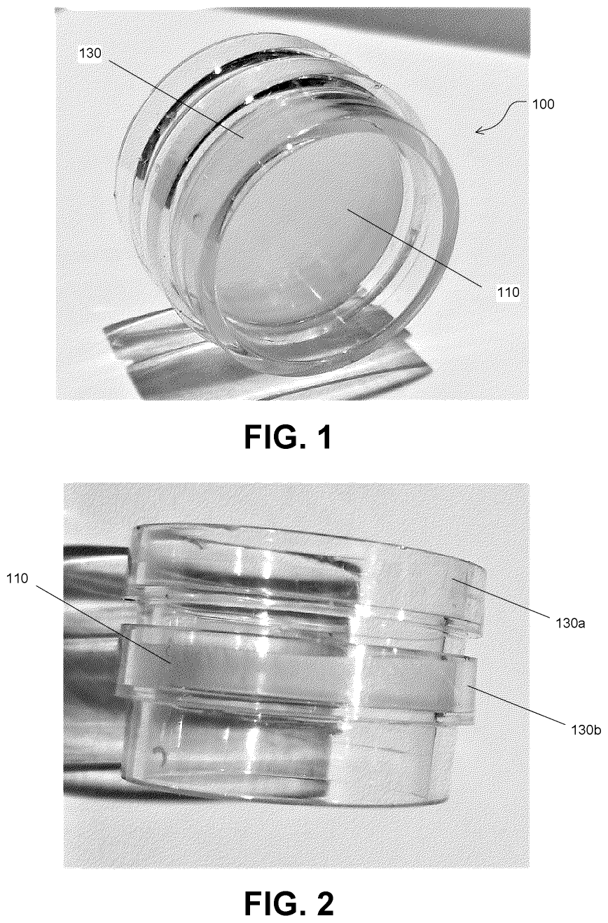

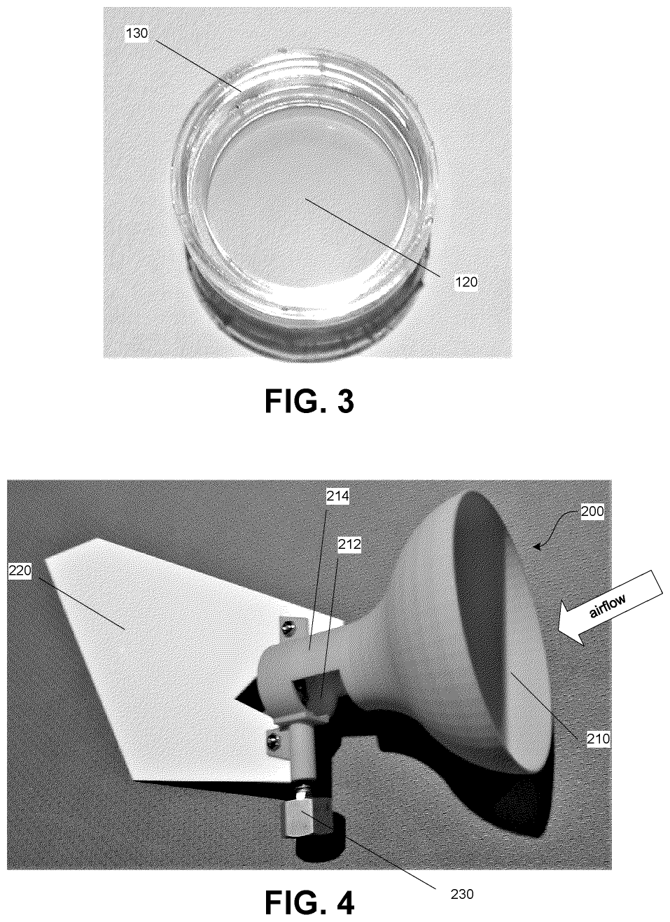

Turning to FIGS. 1 and 3, an embodiment of a pathogen capture device is shown in the form of a cassette 100. The cassette has a collection medium 110 for passive capture of pathogens in the air, and a support frame 120. The support frame 120 supports and keeps the collection medium 110 taut, thereby exposing the collection medium surface 130 to air flow. The collection medium surface 130 allows air to flow through while capturing pathogens in the air.

In one embodiment, the collection medium is made of electrostatically charged fibers. Preferably, the collection medium is a polymer mesh made of electrostatically charged fibers. In some embodiments, the polymer mesh is woven from monofilament fiber. In other embodiments, the polymer mesh is woven from multifilament fiber.

In some embodiments, the polymer mesh is made of a polyester material. In one embodiment, the polymer mesh is made of polyamide, polyethylene, polypropylene, ethylene tetrafluoroethylene, or polyether ether ketone fibers, or a combination of these fibers. In one embodiment, the polymer mesh is made of polyamide.

In some embodiments, the polymer mesh has a mesh size of 1 μm to 200 μm, preferably between 10 μm and 150 μm. In one embodiment, the mesh size is 10 μm, 15 μm, 20 μm, 25 μm, 30 μm, 50 μm, 100 μm, or 150 μm. In some embodiments, the mesh size is selected based on a target pathogen.

Turning to FIG. 2, support frame 120 comprises a pair of mating members 130a, 130b that compression fits together, pinching the collection medium 110 in between to keep the collection medium surface 120 taut and spans the entire area encircled by the pair of mating members. In one embodiment, the pair of mating members are two interlocking rings, having an internal diameter of 0.5 to 3 inches, preferably 1 to 2 inches, more preferably about 1.5 inches. In other embodiments, the pair of mating members are square, polygonal, or other shapes.

In some embodiments, the support frame is also electrostatically charged. In one embodiment, the support frame is made of plastic, for example, styrene or a polystyrene plastic.

The cassette 100 is disposable. Pathogens are captured by the cassette by interception, diffusion, impaction, electrostatic attraction, and/or sedimentation. Although some filtration effect is occurring, this is not the main source of particle/pathogen capture. The collection medium 110 of cassette 100 is also easily removed from the cassette by unlocking the pair of mating members 130a, 130b. The collection medium 110 is then further analyzed using molecular analysis to identify the pathogens collected.

In some embodiments, the collection medium 110 is a mesh and is removed from the cassette and placed directly into a vial for DNA extraction. Bioaerosols such as spores which are bound to the mesh mostly via static attraction are readily released from mesh once a liquid solution is applied. As such, the bioaerosol is not bound to the mesh by any adhesive matrix and therefore does not act as a PCR inhibitor. The mesh is also compatible with standard PCR analysis procedures.

Pathogen Collection and Analysis

In use, the cassette is replaceably inserted into a rotatable wind vane apparatus 200 to direct air to the cassette. As shown in FIG. 4, a wind vane apparatus 200 has a funnel 210, a vane 220, and a post adaptor 230. The funnel 210 concentrates the inflowing air, while the vane 220 directs the funnel based on wind direction. The post adaptor 230 allows the wind vane apparatus 200 to be mounted at the end of a post. When mounted, the wind vane apparatus 200 rotates about the post based on wind direction.

In some embodiment, the wind apparatus does not have a vane and the funnel is positioned based on a desired direction. In some embodiments, the wind apparatus does not have a funnel but has a vane. In other embodiments, the wind apparatus does not have a vane or a funnel.

In some embodiments, the wind apparatus is a drone. In other embodiments, the cassette is placed on a drone, or other vehicle used in agriculture, such as a tractor or truck.

In some embodiments, the wind apparatus has a vane and rotatable about a post. For example, the wind vane apparatus is attached to a standardized plumbing threads of a ½″ MIP fitting or integrated threaded pipe. This allows end users to obtain the pipes of desired length for desired deployment.

In some embodiments the wind vane apparatus has a receptacle for receiving the cassette and the funnel directs flow of air to the capture surface of the cassette. In one embodiment, the cassette is positioned adjacent to the funnel and downstream to a neck portion 214 of the funnel. As used herein, the terms “upstream” and “downstream” are relative to the direction of flow of air. In one embodiment, the cassette is positioned inside the funnel, such as proximate to the upstream end of the funnel, middle of the funnel, or proximate to the downstream end of the funnel, capturing particles and pathogen as air flows through the funnel. In one embodiment, as shown in FIG. 4, the neck portion 214 of the funnel 210 has an opening 212 sized to receive the cassette.

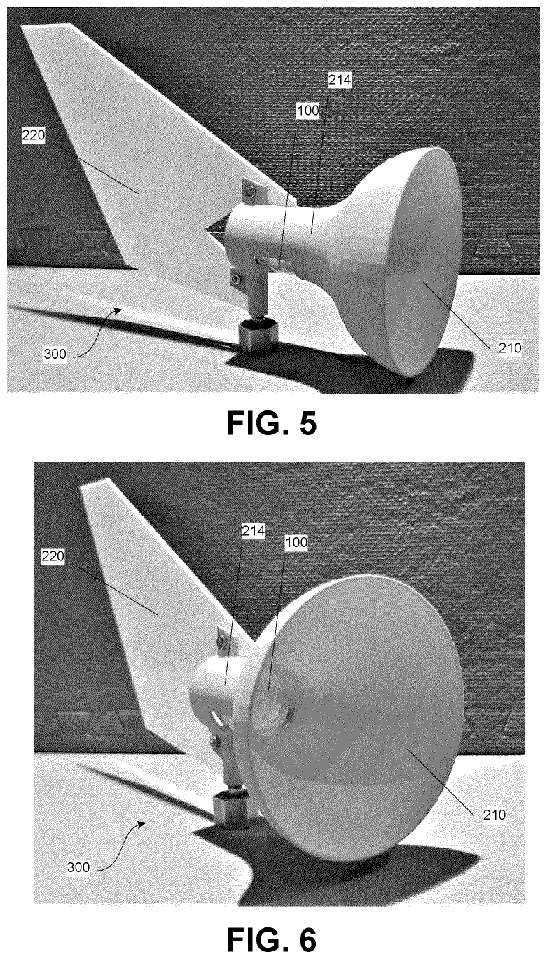

FIGS. 5 and 6 shows a pathogen collection system 300 comprising the wind vane apparatus 200 having a cassette 100 is inserted therein. The cassette is inserted through opening 212 into the neck portion 214 of funnel 210. In one embodiment, the diameter of the cassette corresponds to the inner diameter of the neck portion, such that the collection medium surface 120 spans substantially the full circular cross section of the neck portion, perpendicular to the direction of airflow. In other embodiments, the diameter of the cassette is smaller than the inner diameter of the neck portion, and the collection medium surface 120 partially spans the circular cross section of the neck portion, perpendicular to the direction of airflow.

The cassette is replaced every 1 day, every 2 days, every 3 days, or more. After use, the cassettes are collected for molecular analysis. As used herein, “molecular analysis” refers to analytical techniques including, but not limited to: real-time PCR, conventional PCR, quantitative PCR, multiplex PCR, nested PCR, community sequencing, hi-throughput sequencing, Recombinase Polymerase Amplification (RPA), Loop mediated isothermal amplification (LAMP), antibody/antigen assays, colorimetric assays, or ELISAs. The molecular analysis is used to determine the presence or absence of bioaerosols including pathogens on the cassette. The molecular analysis is used to quantify bioaerosols including pathogens on the cassette

The pathogen collection system is not limited to certain types of pathogens or pathogenic particles (spores, fragments of spores/hyphae) but can passively capture any wind-dispersed pathogenic particle. Furthermore, the system can capture multiple spore types at the same time, and molecular testing on multiple spore types is possible by modifications to a standard PCR cycle to a multiplex PCR cycle.

Spray Decisions

Currently, pesticide spray decisions are often made by growers and agricultural experts based on host susceptibility and environmental factors as a pre-emptive strategy. Information pertaining to the disease-causing pathogen is often only available post-infection by visual scouting of a grower's crop field or by information disseminated from the same strategy in neighbouring fields and regions.

The present disclosure also provides surveillance systems and methods that allows pathogen information to be gathered and made available to growers and agricultural experts prior to infection. Pathogenic particles can be detected in the air before they cause the infection. This allows more information to be considered when deciding when, what and if to spray.

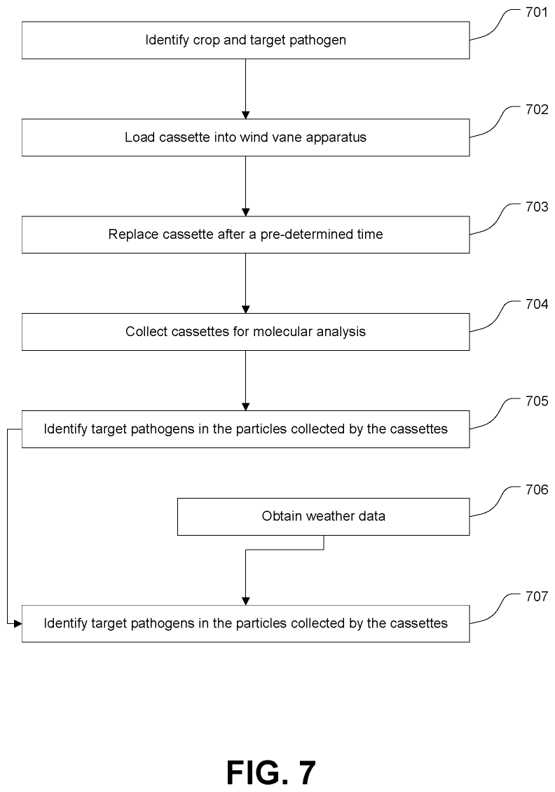

Turning to FIG. 7 the surveillance systems and methods involve first identifying a crop and a target pathogen 701. A wind vane apparatus as described herein is installed and positioned at various heights, depending on the crop and bioaerosol/pathogen, frequently canopy height in a field of crops. It remains in the fields duration the entire growing season or a part of the growing season depending on the crop. Each crop has a window of susceptibility to various pathogens and preferably pathogen collection system described herein is installed at least partially during this window of susceptibility.

When collection of pathogens in the air is desired, a cassette as described herein is loaded into the wind vane apparatus 702. A single or multiple cassettes are used for capturing pathogens. For example, cassettes can be optionally replaced after a pre-determined period of time for maximizing pathogen capture 703. Multiple wind vane apparatuses can be positioned through a crop field to collect pathogens at different locations.

Following pathogen capture, the cassettes are collected for molecular analysis 704. Optionally weather data associated with the time in which pathogen collection was conducted is obtained 706.

Target spores captured by the cassette are differentiated or identified 707 by multiple methods, said methods determining the presence of target organisms yielding a value. For example, the value is numerical, distinctly quantitative, distinctly qualitative or semi-quantitative or semi-qualitative.

This value is then used to determine spray decisions Said determination of spray decisions includes to spray based on presence of the organism, to not-spray based on the presence of the organism, to not-spray based on the absence of the organism, or to spray based on the absence of the organism.

Numerous details are set forth to provide an understanding of the examples described herein. The examples may be practiced without these details. The description is not to be considered as limited to the scope of the examples described herein.

### EXAMPLES

### Example 1—Phytophthora infestans

Looking for Phytophthora infestans (Late blight of Potato) in a Potato field. Potatoes are susceptible to this disease at any time during the life cycle. Therefore the pathogen collection system described above can remain in the field for the entire growing season.

Cassettes are replaced every 3-4 days and sent to the lab for analysis.

### Example 2—Sclerotinia sclerotiorum

Looking for Sclerotinia sclerotiorum (Stem rot of Canola) in Canola Fields. Canola is susceptible to this disease during flowering only. Therefore the pathogen collection system described above can be placed in the field during this time and removed after flowering.

Cassettes are replaced every 2 days during flowering only and sent to the lab for analysis.

Although the embodiments have been described in detail, it should be understood that various changes, substitutions and alterations can be made herein. Moreover, the scope of the present application is not intended to be limited to the particular embodiments or examples described in the specification. As can be understood, the examples described above and illustrated are intended to be exemplary only.

For example, the present invention contemplates that any of the features shown in any of the embodiments described herein, may be incorporated with any of the features shown in any of the other embodiments described herein, and still fall within the scope of the present invention.

What is claimed is:

1. An cassette for collecting bioaerosols, the cassette comprising:
a collection medium, comprising an electrostatically charged fiber; and
a support frame for supporting the collection medium in a extended position to expose a capture surface for capturing bioaerosols.

2. The cassette of claim 1, wherein the collection medium comprises a polymer mesh.

3. The cassette of claim 2, wherein the polymer mesh is comprised of a woven monofilament fiber.

4. The cassette of claim 2 or 3, wherein the polymer mesh is comprised of a polyamide, polyethylene, polypropylene, ethylene tetrafluoroethylene, polyether ether ketone, or combinations thereof.

5. The cassette of claim 4, wherein the polymer mesh is comprised of polyamide.

6. The cassette of any one of claims 2-5, wherein the polymer mesh has a mesh size of 1 μm to 200 μm.

7. The cassette of any one of claims 1-6, wherein the support frame is electrostatically charged styrene.

8. The cassette of any one of claims 1-7, wherein the support frame comprises a pair of mating members configured to secure the collection medium there between when the pair of mating members are coupled together.

9. The cassette of claim 8, wherein the support frame comprises a pair of mating rings.

10. The cassette of claim 9, wherein the collection medium spans across the entire opening area defined by the pair of mating members.

11. The cassette of any one of claims 1-10 for collecting plant pathogens.

12. The cassette of claim 11, wherein the plant pathogens comprise spores.

13. A bioaerosols collection system comprising:
the cassette of any one of claims 1-12; and
a wind apparatus comprising:
a receptacle for receiving the cassette; and
a funnel for directing flow of air to the capture surface of the cassette.

14. The system of claim 13, wherein the receptacle comprises an opening in a neck portion of the funnel for insertion of the cassette.

15. The system of claim 14, wherein the capture surface of the cassette extends at least partially across a cross-section of the neck portion of the funnel.

16. The system of any one of claims 13-15, wherein the wind apparatus comprises a vane for directing the funnel based on wind direction.

17. The system of any one of claims 13-16, comprising a post and wherein the wind apparatus is rotatably mounted on the post.

18. A method of monitoring crops, the method comprising:
identifying a target pathogen;
placing the cassette of any one of claims 1-12 in a wind apparatus for directing flow of air to the capture surface of the cassette;
collecting the cassette;
analyzing the cassette for presence of the target pathogen.

19. The method of claim 18, wherein the cassette is replaced after a pre-determined time, and a plurality of cassettes are collected and analyzed.

20. The method of claim 18 or 19, wherein analyzing the cassette comprises molecular analysis of particles captured by the cassette by real-time PCR, conventional PCR, quantitative PCR, multiplex PCR, nested PCR, community sequencing, hi-throughput sequencing, Recombinase Polymerase Amplification (RPA), Loop mediated isothermal amplification (LAMP), antibody/antigen assays, colorimetric assays, and/or ELISAs.

21. The method of any one of claims 18-20, wherein the method comprises providing a decision based on the presence of the target pathogen.

22. The method of claim 21, wherein the decision comprises a spray decision when presence of the target pathogen is detected.

23. The method of claim 21, wherein the decision comprises a spray decision when presence of the target pathogen absent.

24. The method of any one of claims 21-23, wherein the decision is further based on weather data.
